(19) United States
(12) Patent Application Publication
(10) Pub. No.: US 2021/0361304 A1
(43) Pub. Date: Nov. 25, 2021

(54) Reducing Pulmonary Hypertension with Nitric Oxide Microbubbles

(71) Applicant: Agitated Solutions Inc., Oakdale, MN (US)

(72) Inventor: Micah J. Eimer, Glenview, IL (US)

(73) Assignee: Agitated Solutions Inc., Oakdale, MN (US)

(21) Appl. No.: 17/321,854

(22) Filed: May 17, 2021

Related U.S. Application Data
(60) Provisional application No. 63/026,212, filed on May 18, 2020.

(51) Int. Cl. A61B 17/22; A61M 25/01

(52) U.S. Cl. CPC A61B 17/22; A61M 2005/006; A61B 2017/22089; A61M 25/01

(57) ABSTRACT

A method may include producing, in a solution, microbubbles of nitric oxide having a targeted size; and directing the solution to a right side of a patient's heart, such that the microbubbles are directed to the patient's pulmonary arteries prior to being circulated through the patient's body. The solution may include saline or dextrose. Directing the solution to the right side of the patient's heart may include delivering the solution via a catheter, during a right-heart catheterization procedure. Directing the solution to the right side of the patient's heart may include injecting the solution into a median cubital vein of the patient. Directing the solution to the right side of the patient's heart comprises injecting the solution into an internal jugular vein, an external jugular vein or a femoral vein of the patient. The targeted size may correspond to a diameter of a specific branch of the patient's pulmonary arteries.

### CROSS-REFERENCE TO RELATED APPLICATIONS

This application claims the benefit of U.S. Provisional Application Ser. No. 63/026,212, titled “Reducing Pulmonary Hypertension with Nitric Oxide Bubbles,” filed on May 18, 2020. This application incorporates the entire contents of the foregoing application herein by reference.

### TECHNICAL FIELD

Various embodiments relate generally to treating pulmonary hypertension.

### BACKGROUND

Nitric oxide is a natural vasodilator. When inhaled, it can be used to treat persistent pulmonary hypertension of the newborn (PPHN); and it can be used in adults with pulmonary arterial hypertension (PAH).

### SUMMARY

In some implementations, a method includes producing, in a solution, microbubbles of nitric oxide having a targeted size; and intravenously directing the solution into a patient's venous circulatory system and to the right side of the patient's heart, such that the microbubbles are directed to the patient's pulmonary arteries prior to being circulated through the patient's body.

The solution may include one of saline or dextrose. Intravenously directing the solution may include injecting the solution into a median cubital vein of the patient. Intravenously directing the solution may include injecting the solution into an internal jugular vein, an external jugular vein or a femoral vein of the patient.

The targeted size may correspond to a diameter of a specific branch of the patient's pulmonary arteries. In some implementations, the targeted size includes one of 0.056±0.005 mm, 0.036±0.005 mm or 0.020±0.003 mm. In some implementations, the targeted size includes one of 0.34±0.06 mm, 0.22±0.02 mm, 0.15±0.02 mm, or 0.097±0.012 mm. In some implementations, the targeted size includes one of 1.16±0.10 mm, 0.77±0.07 mm or 0.51±0.04 mm.

In some implementations, a method includes producing, in a solution, microbubbles of nitric oxide having a targeted size; and directing the solution to a right side of a patient's heart, such that the microbubbles are directed to the patient's pulmonary arteries prior to being circulated through the patient's body.

In some implementations, the solution includes saline or dextrose. Directing the solution to the right side of the patient's heart may include delivering the solution via a catheter, during a right-heart catheterization procedure. Directing the solution to the right side of the patient's heart may include injecting the solution into a median cubital vein of the patient. Directing the solution to the right side of the patient's heart comprises injecting the solution into an internal jugular vein, an external jugular vein or a femoral vein of the patient.

The targeted size may correspond to a diameter of a specific branch of the patient's pulmonary arteries. In some implementations, the targeted size includes one of 0.056±0.005 mm, 0.036±0.005 mm or 0.020±0.003 mm. In some implementations, the targeted size includes one of 0.34±0.06 mm, 0.22±0.02 mm, 0.15±0.02 mm, or 0.097±0.012 mm. In some implementations, the targeted size includes one of 1.16±0.10 mm, 0.77±0.07 mm or 0.51±0.04 mm.

### BRIEF DESCRIPTION OF THE DRAWINGS

FIG. 1 illustrates a portion of a human circulatory system.

FIG. 2 illustrates detail of a human heart.

FIG. 3A illustrates various aspects of a human respiratory system.

FIG. 3B illustrates detail associated with a portion of a human pulmonary system.

FIG. 3C illustrates detail associated with human pulmonary arteries.

FIG. 4 depicts an exemplary method for reducing pulmonary hypertension in a patient.

### DETAILED DESCRIPTION

Hypertension, or high blood pressure, can cause a variety of significant health problems, including various forms of heart disease (e.g., coronary artery disease, enlarged left heart, heart failure), various injuries to the brain (e.g., transient ischemic attack (TIA), stroke, mild cognitive impairment, dementia), and damage to other organs (e.g., kidney scarring or failure, retinopathy, choroidopathy, optic neuropathy).

Blood pressure is determined by the amount of blood pumped by the heart and the resistance to blood flow in the arteries. Either more blood, or narrower, more rigid arteries, or both, increases blood pressure. While exact causes of high blood pressure are not fully understood, there are a number of risk factors, including smoking, obesity, dietary factors (e.g., too much salt or alcohol), stress, age, genetics, sleep apnea, or other underlying health conditions (e.g., chronic kidney disease, adrenal and thyroid disorders, sleep apnea).

Pulmonary hypertension (PHT) is high blood pressure in the arteries of the lungs. Patients with PHT often have hard and narrow vessels that carry blood from the heart to the lungs, forcing the heart to work harder. Such patients may experience shortness of breath, fatigue, chest pain, dizziness, or fainting.

There is no cure for PHT, but certain treatments can reduce symptoms and help manage the condition. Left untreated, PHT can lead to swelling of the legs, blood clots, difficulty breathing or heart failure.

PHT may be classified as either secondary or primary. Secondary PHT stems from other underlying conditions that result in vasoconstriction (the constriction of blood vessels, increasing blood pressure), such as emphysema and other forms of chronic obstructive pulmonary disease (COPD), collagen vascular diseases (e.g., scleroderma, CREST syndrome and lupus), congenital heart disorders (e.g., ventricular and septal defects), blood clots in the lungs and pulmonary arteries, HIV, liver diseases, diet drugs (e.g., fenfluramine, dexfenfluramine).

Primary PHT (defined as pulmonary arterial pressure greater than 25 mm Hg at rest or greater than 30 mm Hg during exercise) has unknown causes; and the condition is very serious. Left untreated, prognosis is poor and the disease can be fatal within a few years; though survival rates for patients with primary PHT have been steadily increasing as new drugs and therapies have become available.

In acute settings, vasodilators—substances that dilate or open blood vessels, allowing blood to flow more easily—can have beneficial effect on patients with PHT. One natural vasodilator is nitric oxide. In certain settings, nitric oxide can be inhaled to acutely treat certain forms of PHT. For example, inhaled nitric oxide can be used to treat persistent pulmonary hypertension of the newborn (PPHN); similarly, it may be used in some adults with PHT.

Inhaled nitric oxide is not without risks, however. First, inhalation of nitric oxide brings about a systemic effect, rather than one that is focused on the pulmonary arteries. This is because of where the transfer of nitric oxide to the body occurs. When inhaled, nitric oxide is absorbed by pulmonary veins that return oxygenated blood to the heart, for distribution to the rest of the body. Thus, to reach the pulmonary arteries—the vessels that are thought to be most influential in causing PHT—nitric oxide that is absorbed through inhalation circulates throughout the body before reaching the pulmonary arteries on a return circuit through the lungs. This causes non-specific vasodilation—which can create its own issues if high blood pressure is focused primarily in the lungs.

A second risk with inhaled nitric oxide is that it can become toxic in or damaging to the lungs. For example, nitric oxide (NO) can rapidly react with oxygen to form nitrogen dioxide, which is a potent pulmonary irritant; specifically, nitrogen dioxide can oxidize protective antioxidants within the epithelial lining fluid (ELF), triggering extracellular damage in the airways. As another example, nitric oxide can also react with superoxide anion to form peroxynitrate, which is a cytotoxic oxidant that can interfere with surfactant functioning in the lungs. For these reasons, inhalation of nitric oxide as a vasodilator to treat PHT is not ideal.

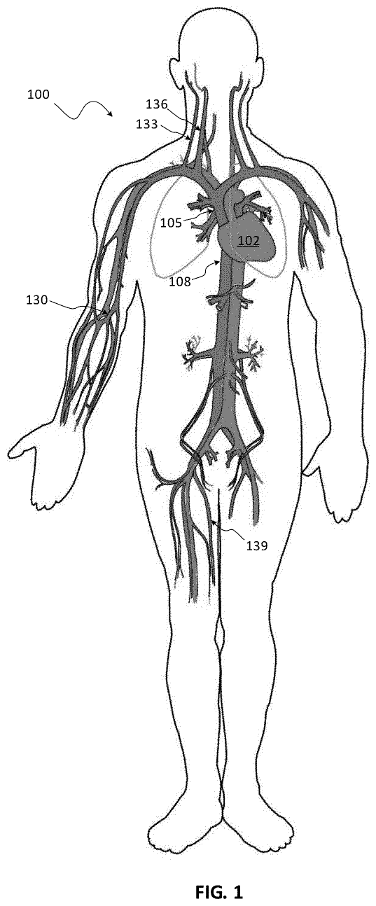

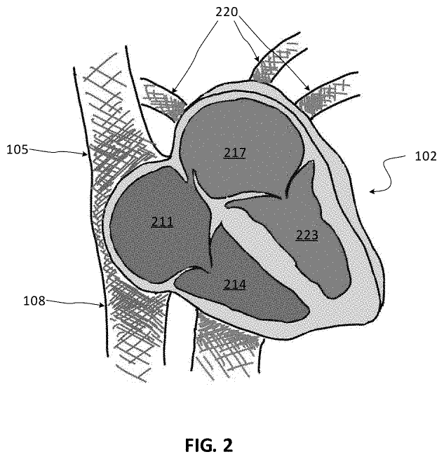

Described now is another delivery mechanism for nitric oxide that is more targeted to the pulmonary arteries—a believed focus of PHT. For context, various aspects of a human cardiovascular system are first described with reference to FIG. 1 and FIG. 2. FIG. 1 illustrates a portion of an overall human circulatory system 100. At its core is the heart 102, and a system of arteries that extend from the heart, and veins that return to the heart.

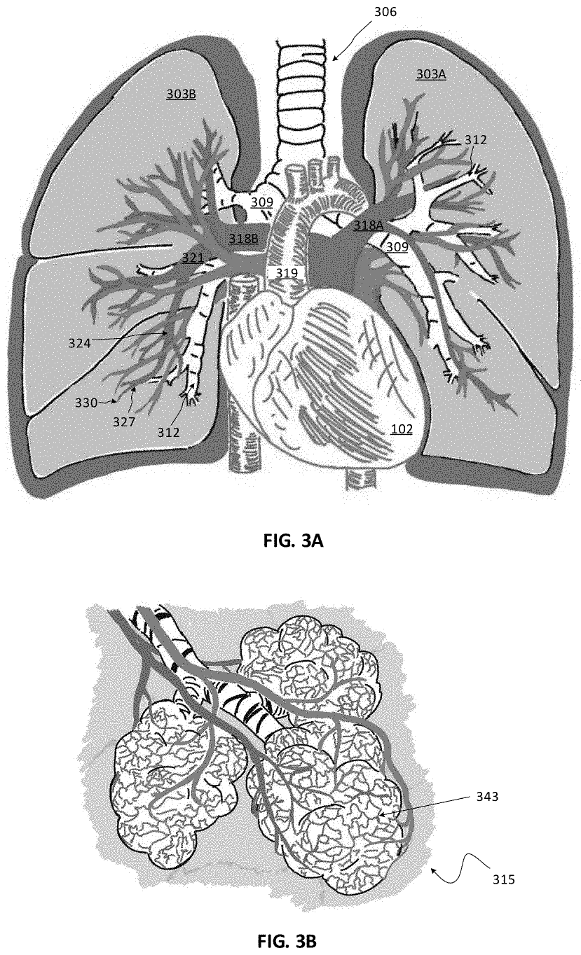

Various internal structures of the heart 102 are described in greater detail with reference to FIG. 2. Blood is returned to the heart 102 from throughout the body by the vena cava, which is divided into the superior vena cava 105, which collects blood from the upper portion of the body, and the inferior vena cava 108, which collects blood from the lower portion of the body. Blood flows through the superior vena cava 105 and inferior cava 108 on its way to the right atrium 211. From the right atrium 211, the heart 102 pumps blood into the right ventricle 214; the right ventricle 214 pumps blood to the lungs 303A and 303B, via pulmonary arteries 318A and 318B (see FIG. 3A).

After being oxygenated in the lungs, blood is returned to the left atrium 217 of the heart 102 via the pulmonary veins 220 (three of four of which are shown). From the left atrium 217, the heart 102 pumps blood into the left ventricle 223, which in turns pumps it to the aorta 319 for distribution throughout the body.

In some instances, it may be advantageous to introduce therapeutic or diagnostic substances into the human circulatory system. For example, clinicians may conduct “bubble studies,” whereby they may introduce agitated saline or dextrose (saline or dextrose with microbubbles, which can serve as a contrast agent for imaging modalities, such as ultrasound). Alternatively, clinicians may generate microbubbles using therapeutic gases or compounds (e.g., nitric oxide) to deliver targeted therapeutic benefit to a patient.

To facilitate such bubble studies or other diagnostic or therapeutic procedures whereby microbubbles are to be introduced into the heart and lungs, one must get the microbubbles into the venous circulatory system and ultimately into the superior vena cava 105 or inferior vena cava 108, and into the right atrium 211 of the heart 102. With reference to FIG. 1, there are several common access points through which microbubbles can be introduced. Common among them is intravenous introduction of microbubbles (e.g., mixed with saline or dextrose) via the median cubital vein 130 of the arm. From here, blood flows through the basilic vein, axillary vein, subclavian vein, and into the superior vena cava 105. Alternative paths to the superior vena cava 105 are the external jugular vein 133, or internal jugular vein 136, both of which drain into the brachiocephalic vein prior to reaching the superior vena cava 105. An alternative inferior route includes the femoral vein 139, which flows into the inferior vena cava 108 prior to reaching the right atrium 211. Other routes to the right atrium 211 are possible.

FIG. 3A, FIG. 3B and FIG. 3C illustrate various aspects of the respiratory and pulmonary systems. The lungs 303A and 303B receive air through a progressively smaller network of airways that include the trachea 306, bronchi 309, bronchioles 312 and alveoli 315. A corresponding network of progressively smaller pulmonary arteries, including the arteries 318B, 321, 324, 327, 330, 333, 336 and even smaller vessels (not shown) deliver deoxygenated blood ultimately to alveoli capillaries 343, disposed on the thin walls of individual alveoli 315. Here, oxygen is passed to the blood, and carbon dioxide is released from the blood. Here is also where other gases present in the lungs are absorbed (e.g., inhaled nitric oxide). From the alveoli 315, a progressively larger network of veins carries oxygenated blood back to the heart 102—specifically, the left atrium 217, via the pulmonary veins 220.

FIG. 3C illustrates additional detail associated with a pulmonary artery 317. As shown, the pulmonary artery 317 branches and decreases in size as it extends into the lung 303B, from the heart 102. Branches include the right pulmonary artery 318B and progressively smaller branches 321, 324, 327, 330, 333, 336, and smaller (not shown), each of which as a decreasing diameter.

In some patients, pulmonary artery branches range in size as follows, from largest-diameter branch to smallest-diameter branch: 14.80±2.10 mm, 7.34±1.14 mm, 4.16±0.60 mm, 2.71±0.35 mm, 1.75±0.19 mm, 1.16±0.10 mm, 0.77±0.07 mm, 0.51±0.04 mm, 0.34±0.06 mm, 0.22±0.02 mm, 0.15±0.02 mm, 0.097±0.012 mm, 0.056±0.005 mm, 0.036±0.005 mm, and 0.020±0.003 mm.

As depicted in FIG. 3C, microbubbles that are introduced intravenously (e.g., via the median cubital vein 130) can reach a lung 303B, via the network of pulmonary arteries 317. Given their very small size, microbubbles are typically absorbed in the pulmonary capillaries 343 (see FIG. 3B), if not before. However, by varying the size of the microbubbles, it may be possible to “target” specific portions of the pulmonary arteries (e.g., segments 336, 333, 330, 324, etc.), prior to the microbubbles reaching the pulmonary capillaries 343.

By producing such microbubbles from nitric oxide, it may be possible to target delivery of nitric oxide to specific portions of the pulmonary arteries, as a vasodilator (e.g., for treatment of PHT)—in some cases, without the deleterious effects noted above. That is, such targeting may result in localized vasodilation, rather than systemic vasodilation. Moreover, since the nitric oxide may be delivered directly to the cell wall of pulmonary arteries—in the absence of oxygen in the alveoli (as in the case of inhaled nitric oxide)—it may be possible to avoid or minimize the generation of nitrogen dioxide or other toxic nitrogen-containing compounds. Furthermore, delivery to the cell wall of a pulmonary artery may have the effect of directly dilating the smooth muscle comprising a portion of the artery wall, which smooth muscle, when constricted, may contribute to PHT.

By modulating the size of the microbubbles through the process through which they are produced, it may be possible to target very specific portions of the pulmonary arteries—e.g., portions that are believed to contribute most to PHT. For example, larger microbubbles 350 may be used to target larger-diameter portions of the pulmonary arteries 317 (e.g., branch 324); medium sized microbubbles 353 may be used to target smaller-diameter portions of the pulmonary arteries 317 (e.g., branch 330); smaller microbubbles 356 may be used to target still smaller-diameter portions of the pulmonary arteries 317 (e.g., branches 333 or 336).

Note that FIG. 3C is merely representative of a branching structure whose branch diameters are progressively smaller the farther out they are. For purposes of illustrating targeting specific portions of the pulmonary arteries, branch 336 may depict the smallest, terminal branch, at a patient's alveoli (e.g., a branch having a diameter of about 0.020±0.003 mm)(where “about” means within a specified tolerance or within 1%, 5%, 10%, 20%, 25% or 50% of a nominal value); and in an actual patient, there may be many additional branches between 336 and the preceding branches 333, 330, 327, 324, 321 and/or 318.

Other delivery mechanisms of nitric oxide microbubbles may be employed in other procedures without deviating from the teachings herein. For example, nitric oxide microbubbles may be delivered in the context of a right heart catheterization (e.g., via a catheter used in the procedure) to evaluate pulmonary vasoreactivity. That is, rather than injecting saline with nitric oxide microbubbles into a vein as described above, saline with nitric oxide microbubbles may be delivered to a specific part of the heart or pulmonary circuit via a lumen in a catheter.

Prior to delivery of the nitric oxide microbubbles, baseline atrial pressure, ventricle pressure, pulmonary artery pressure, pulmonary wedge pressure, and other physiological parameters may be captured; and subsequent to delivery of the nitric oxide microbubbles, the same pressure and other parameters may again be captured and compared to the baselines to determine vasoreactivity.

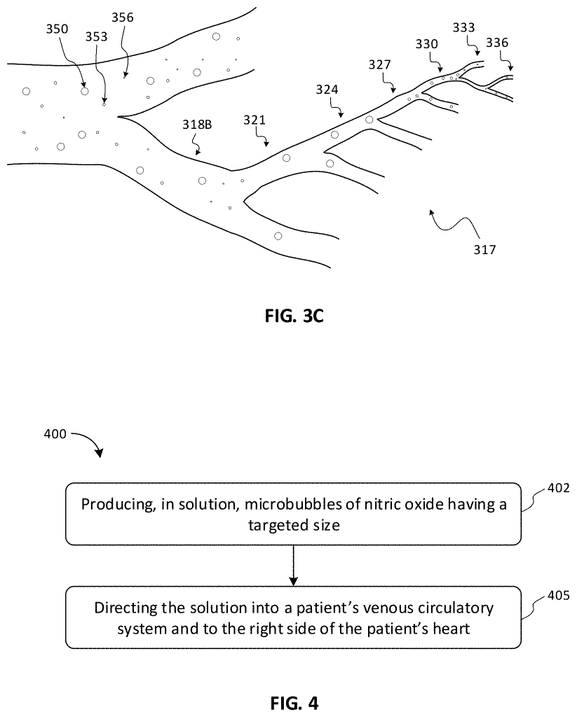

FIG. 4 depicts an exemplary method 400 for reducing pulmonary hypertension in a patient. As shown, the method 400 includes producing (402), in a solution, microbubbles of nitric oxide having a targeted size. In some implementations, the solution is either saline or dextrose; in other implementations, the solution comprises another body-compatible fluid.

The method 400 further includes directing (405) the solution into a patient's venous circulatory system and to the right of side of the patient's heart. In some implementations, the solution is injected intravenously, for example, through the patient's median cubital vein (e.g., vein 130, shown in FIG. 1.). In other implementations, the solution is injected intravenously through another vein, such as the internal jugular vein 136, external jugular vein 133 or femoral vein 139. In still other implementations, the solution may be more directly delivered to the patient's right heart, for example through a catheter during a right-heart catheterization procedure.

Regardless of the specific site or manner of injection, the solution may directed into the patient's venous circulatory system in a manner that causes the microbubbles in the solution to reach the patient's pulmonary arteries prior to being circulated through the patient's body. In this manner, a more tailored vasodilation of the pulmonary arteries may be achieved, rather than the systemic effect that may result when a vasodilator such as a nitric oxide is introduced downstream, such as in the pulmonary veins (e.g., as is the case with inhaled nitric oxide).

As described above, size of the microbubbles may be tailored to correspond to specific portion(s) of the patient's pulmonary artery network, in order to provide vasodilation therapy where it may be most beneficial, given the patient's history, health condition, past injuries, etc.

While several implementations have been described with reference to exemplary aspects, it will be understood by those skilled in the art that various changes may be made and equivalents may be substituted for elements thereof without departing from the contemplated scope. For example, nitric oxide microbubbles may be sized to target different branches of a patient's pulmonary arteries. Exemplary branch sizes are provided as references, but microbubble size may be modulated to account for deviations from the representative ranges for age or gender of a specific patient. Moreover, for some patients, different portions of the pulmonary arterial circuit may be targeted based on health history, health condition or prior injury to the patient being treated. Many modifications may be made to adapt a particular situation or material to the teachings provided herein without departing from the essential scope thereof. Therefore, it is intended that the scope not be limited to the particular aspects disclosed but include all aspects falling within the scope of the appended claims.

What is claimed is:

1. A method comprising:
producing, in a solution, microbubbles of nitric oxide having a targeted size; and
intravenously directing the solution into a patient's venous circulatory system and to the right side of the patient's heart, such that the microbubbles are directed to the patient's pulmonary arteries prior to being circulated through the patient's body;
wherein, the targeted size corresponds to a diameter of a specific branch of the patient's pulmonary arteries.

2. The method of claim 1, wherein the solution comprises one of saline or dextrose.

3. The method of claim 1, wherein intravenously directing the solution comprises injecting the solution into a median cubital vein of the patient.

4. The method of claim 1, wherein intravenously directing the solution comprises injecting the solution into an internal jugular vein, an external jugular vein or a femoral vein of the patient.

5. The method of claim 1, wherein the targeted size comprises one of 0.056±0.005 mm, 0.036±0.005 mm or 0.020±0.003 mm.

6. The method of claim 1, wherein the targeted size comprises one of 0.34±0.06 mm, 0.22±0.02 mm, 0.15±0.02 mm, or 0.097±0.012 mm.

7. The method of claim 1, wherein the targeted size comprises one of 1.16±0.10 mm, 0.77±0.07 mm or 0.51±0.04 mm.

8. A method comprising:
producing, in a solution, microbubbles of nitric oxide having a targeted size; and
directing the solution to a right side of a patient's heart, such that the microbubbles are directed to the patient's pulmonary arteries prior to being circulated through the patient's body;
wherein, the targeted size corresponds to a diameter of a specific branch of the patient's pulmonary arteries.

9. The method of claim 8, wherein directing the solution to the right side of the patient's heart comprises delivering the solution via a catheter, during a right-heart catheterization procedure.

10. The method of claim 8, wherein directing the solution to the right side of the patient's heart comprises injecting the solution into a median cubital vein of the patient.

11. The method of claim 8, wherein directing the solution to the right side of the patient's heart comprises injecting the solution into an internal jugular vein, an external jugular vein or a femoral vein of the patient.

12. The method of claim 8, wherein the solution comprises one of saline or dextrose.

13. The method of claim 8, wherein the targeted size comprises one of 0.056±0.005 mm, 0.036±0.005 mm or 0.020±0.003 mm.

14. The method of claim 8, wherein the targeted size comprises one of 0.34±0.06 mm, 0.22±0.02 mm, 0.15±0.02 mm, or 0.097±0.012 mm.

15. The method of claim 8, wherein the targeted size comprises one of 1.16±0.10 mm, 0.77±0.07 mm or 0.51±0.04 mm.
